(19) United States
(12) Patent Application Publication
(10) Pub. No.: US 2009/0117170 A1
(43) Pub. Date: May 7, 2009

(54) SEGMENTED DEVICE FOR THE DELAYED RELEASE OF MOLECULES IN A TANGENTIAL DIRECTION THROUGH THIN FILMS AND USES THEREOF

(75) Inventors: Lutz Kroehne, Berlin (DE); Andreas Voigt, Berlin (DE)

Correspondence Address: PATTERSON & SHERIDAN, L.L.P., 3040 POST OAK BOULEVARD, SUITE 1500, HOUSTON, TX, 77056, US

(73) Assignee: CAPSULUTION NANOSCIENCE AG, Berlin (DE)

(21) Appl. No.: 11/577,175

(22) PCT Filed: Oct. 12, 2005
(86) PCT No.: PCT/EP05/10971

§ 371 Date: Dec. 24, 2008

(30) Foreign Application Priority Data
Oct. 12, 2004 (DE) 10 2004 050 457.1

(51) Int. Cl. A61F 2/04; A61M 5/00; B32B 38/18; A61K 9/00

(52) U.S. Cl. 424/424; 604/19; 424/400; 156/182

(57) ABSTRACT

The present invention of a segmented release device (sandwich construction with reservoir) for molecules (active compounds, medicaments, diagnostic, therapeutic and chemical reagents) is based on a construction which makes possible a constant release rate through diffusion-permeable intersegment films partially or completely filled with liquid of the neighboring media. The molecules pass here from the reservoir of the device into the outer medium by diffusion exclusively through the intersegment films. These intersegment films are adjustable in their thickness and composition in the manner specified in each case.

The release rate can be determined in advance and thus also calculated within wide limits by the structure and geometry of the segmented device and by the number, composition and dimensions of the thin intersegment films. The device according to the present invention allows the adjustment of an extremely precise release rate of the molecules. By means of this, an optimal adaptation of the release rate per prescription present in each case can be carried out. On the part of the molecule, only the knowledge of the solubility and of the diffusion coefficient is of outstanding importance here.

Directional dependence of the release is achievable by the shape of segments. Here, areas which are nearer to the reservoir have a shorter diffusion path than areas having a greater distance.

The release capacity can approximately reach the capacity of the reservoir.

### BACKGROUND OF THE INVENTION

This invention relates to a device for the constant release of molecules, in particular pharmaceutical active compounds. The efficiency of the administration of pharmaceutical active compounds in many cases depends very significantly on the form of administration and the administration route. Often, a simple form of administration, e.g. by means of oral absorption as a tablet or liquid, is achieved at the expense of a high intake with numerous side effects and a suboptimal active compound distribution in the body. The administration route is important for the efficacy of many medicaments. It can be a great advantage to have an administration system having a device for administration which releases the active compounds at a controlled rate in the vicinity of the sites of action over a prolonged period. For this reason, implantable administration systems have been developed which can administer the active compounds more safely, more efficiently, more accurately targeted, more lastingly and more reliably (cf., for example, EP 0 914 092; U.S. Pat. No. 6,464,687; U.S. Pat. No. 6,494,867; U.S. Pat. No. 5,085,656; U.S. Pat. No. 6,464,671; U.S. Pat. No. 6,444,217; U.S. Pat. No. 6,309,380; U.S. Pat. No. 5,660,848; U.S. Pat. No. 3,625,214; U.S. Pat. No. 3,854,480; U.S. Pat. No. 3,926,188; U.S. Pat. No. 3,832,252; U.S. Pat. No. 3,948,254; U.S. Pat. No. 3,993,072; U.S. Pat. No. 4,244,949; U.S. Pat. No. 4,639,244; U.S. Pat. No. 4,666,704; U.S. Pat. No. 4,957,119; U.S. Pat. No. 5,035,891; U.S. Pat. No. 5,141,748; U.S. Pat. No. 5,150,718; GB 2 136 688; U.S. Pat. No. 4,786,501; U.S. Pat. No. 5,041,107; U.S. Pat. No. 6,767,550; U.S. Pat. No. 6,743,204; U.S. Pat. No. 6,726,920; DE 101 61 078; U.S. Pat. No. 6,491,683; U.S. Pat. No. 6,086,908; US 20040176749; U.S. Pat. No. 4,601,893; DE 36 05 664; WO 02/100455).

Among the implantable active compound administration systems, there are biodegradable and non-biodegradable systems, in addition systems having constant and variable release rates. Furthermore, active systems are to be distinguished from passive systems. The former release the active compound by utilization of an additional energy source, e.g. osmotically, mechanically or electrically. The passive systems control the release by the diffusion of the active compound from the either stable or degradable implant.

The control of the release rate by diffusion from a stable non-biodegradable, non-swellable implant device, besides the disadvantage of an explanation or new filling of the implant which is usually necessary after a certain time, has a number of advantages, which result from the precise character of the implant construction and its invariability over an arbitrarily long time horizon, the release kinetics playing a crucial role. As a rule, an extremely small interaction exists between the implant and the active compounds. In contrast to this, biodegradable implantable active compound carriers must regularly be tailored to the properties of the specific active compounds in an involved manner in order that reliable release profiles can be achieved.

It is therefore the aim of the present invention to make available a device whose geometrical dimensions and construction essentially determines the release characteristics of the active compounds enclosed in a reservoir in this device. For the construction of the release device, the chemical properties of the active compounds play a subordinate role here. The solubility in the reservoir and the diffusion coefficient of the active compound in the release device are important.

### DETAILED DESCRIPTION

Against this background, a segmented device for the release of molecules or substances is proposed, which contains:
at least two segment discs stacked one above the other having at least one cavity or passage opening, which forms at least one inner reservoir for the molecules or substances;
at least one permeable intersegment film between the segment discs, through which the release of the molecules from the reservoir takes place exclusively; and
means for the holding and fixing of the segment discs.

Intersegment films in the context of the invention should be understood as meaning the intermediate space between adjacent segment discs. This intermediate space can be formed by a separate film. Intersegment films are then formed by films applied to or produced separately on the segment discs, which consist of a different material than the segment discs. In particular, the separate films make possible diffusion of the molecules or substances contained in the reservoir into the environment. Intersegment films, however, can also be formed by “hollow” intermediate spaces between adjacent segment discs which are squeezed or pressed onto one another. The intermediate space is then not filled by a further separate material or separate film. However, the solution medium contained in the reservoir or the solution medium of the environment can wet the segment discs and thereby fill the intermediate space. “Hollow” intersegment films of this type are made possible by segment discs having a predetermined surface roughness. The surface roughness of the segment discs results in the segment discs not completely being able to seal the intermediate space between them, but microscopic cavities remaining which make possible diffusion of the molecules or substances contained in the reservoir. The achievable diffusion rate can be adjusted within wide ranges by the choice of the surface roughness.

The thickness range of the intersegment films (separate film or cavity) can be between 1 nm and 50 μm, preferably between 2 nm and 20 gm and particularly preferably between 10 μm and 1 μm. The preferred average roughness of the segment discs should be less than 250 nm.

Molecules or substances to be released are understood in particular as meaning active compounds, pharmaceuticals, diagnostic, therapeutic and chemical reagents. Molecules or substances of this type can be dissolved, for example, in a suitable solvent in the reservoir. Preferably, sparingly or poorly soluble molecules or substances and in particular sparingly or poorly water-soluble molecules or substances are used. Whether a substance is sparingly soluble or not depends on the type of solvent, which is tailored to the respective intended use and in particular to the surrounding medium into which the substance is to be released. On account of the low solubility of the sparingly soluble substances, these substances are mainly present in a saturated concentration, i.e. constant concentration. Thus, constant release rates are achieved in the case of a diffusion release over long periods of time.

The release of the molecules or substances (active compounds, medicaments, diagnostic, therapeutic and chemical reagents) takes place by diffusion through the thin intersegment films bordered or bounded by the segment discs. The device according to the present invention allows the adjustment of an extremely precise release rate of the molecules. The number, the construction and the dimensions of the intersegment films essentially determine the release rate. Very small rates can be achieved by using very thin intersegment films. This is afforded, as a rule, on the use of very smooth segment discs, e.g. of wafer quality, which have roughnesses in the single-figure nanometre range. Thin intersegment films of this type simultaneously prevent the penetration of relatively large biogenic molecules from the side of the biological medium into the reservoir. Typical examples for the selection of the segment disc materials are all biocompatible substances, e.g. from the classes consisting of the stainless steels, of titanium, of the ceramics, of glasses and of the plastics, further metals, e.g. from the classes consisting of the noble metals, and further inorganic biologically inert solids. The production of the segment discs themselves and the processing of their surfaces are carried out using suitable processes, as, for example, for the processing of semiconductor and wafer surfaces, glass surfaces, ceramic surfaces and polymer film surfaces. It is thus possible to structure the surfaces of the segment discs. A further possibility consists in the construction of adsorption layers and multiadsorption layers, generally of intersegment films, on the segment disc surfaces. The distance of the segment discs one below the other can thus be controlled and varied. Processes in the modification of relatively smooth segment disc surfaces (roughnesses below 1 μm) which suggest themselves for this purpose are, for example, the “Layer-by-Layer” process (Handbook of Polyelectrolytes and their Applications, Volume 1, Tripathy S K, Kumar, J, Nalwa, H S (editors), American Scientific Publishers, Stevenson Ranch, California, 2002; Multilayer Thin Films, Decher G, Schlenoff J B (editors), Wiley-V C H, Weinheim, 2003), in which a sequential adsorption of differently charged polymeric polyelectrolytes or nanoparticles takes place from the aqueous phase. Here, in the invention presented, the selection of the polyelectrolytes and nanoparticles, provided they fulfill their function in the arrangement, is only subjected to the regulatory orders of the respective application areas.

The construction of intersegment film layers penetrable for the molecules from the organic phase or from the exchange of aqueous and organic phase can also take place.

The intersegment film thickness is determined either by the roughness of the segment discs and/or by the surface structuring and/or by the constructed porous and permeable thin film phases. In the case of relatively large intersegment film thicknesses above a few micrometres, the construction of the film phases can be carried out by the layer-by-layer process. Other processes can likewise be used here. For instance, polyelectrolyte complexes can be applied to the discs as a substance in the form of a film. By means of the assembly of the segment discs to give the device, the intersegment film thickness can be adjusted by the pressure of the discs on one another. The excess material is squeezed out in this process and can be removed from the reservoir and from the external medium before application. The application of the intersegment films to the segment discs can also be carried out, for example, by spin-coating or other coating processes developed in polymer chemistry, e.g. spraying, vapor deposition, immersion. The segment discs can be coated here with a defined layer, which in this form and with these measurements can then also be used in the segmented device.

The constructed intersegment films between the segment discs should here be permeable for the release of the molecules from the inside outwards and to the greatest extent impermeable for the penetration of biogenic macromolecules from the outside inwards. Segmented release devices of hydrophobic segment discs, e.g. of Teflon or polyethylene, can be greatly influenced in their release rate by the construction of hydrophilic intersegment films.

The segmented release device is fixed in its arrangement by a holder and closed without central openings and core drillings by means of base and cover segments. The closing mechanism used can be, for example, screwing together, gluing, clamping, welding, wedging, joining. In certain embodiments of the devices, e.g. toroidal arrangement of the segments, the fixing of the device can also be carried out in another form, e.g. without base and cover discs. Magnetic forces can also be used for fixing and closing.

Before the closing of the release device or afterwards, the supply of the formulation containing the active compound (molecules or substances to be released) into the reservoir takes place, e.g. by means of a small sealable opening. Attention should be paid here, inter alia, to filling which is as air bubble-free as possible. The formulation should be adapted to the purpose of administration, the type of release and the chemical or physicochemical properties and conditions of the molecular species and of the material of the segmented device. Release should take place in the dissolved or fluid state. The formulation in the reservoir of the segmented device can be solid, gelatinous or liquid, and can be present as an emulsion or suspension, as a gel or as a solid phase in equilibrium with the saturated solution. A number of molecular species can be enclosed in the device in identical or different formulations. The reservoir can consist of an opening or core drilling connected to one another. It can also consist of a number of sub-reservoirs not connected to one another. All reservoirs, however, must be in direct contact with the intersegment films. The molecules are released through the intersegment films by means of diffusion.

In the case of poorly water-soluble substances, the release devices can achieve constant releases for a number of years, but also exhaustion of the reservoir after one week depending on the chosen geometrical and intersegment film parameters of the device. The quantitative release kinetics result—without wishing to be restricted—in a good approximation of the application of the diffusion laws (e.g. 1st and 2nd Fick's law, Knudsen diffusions). Here, the simple segmented geometry and the control of the intersegment film permeability between the segment discs allow the adjustment of the release behavior within very wide limits. It is therefore possible in advance to be able to estimate or to calculate and to optimize the release behavior on the basis of the proposed construction principles and of the passive diffusive substance transport. Thus, very highly water-soluble molecular species can also be released over very long periods of time if operation is carried out using very smooth discs with thin intersegment film thicknesses or using a small number of intersegment films and at the same time a large reservoir.

The release capacity can approximately reach the capacity of the reservoir. The proportion of the active compound volume to the total volume of the release device can maximally correspond to the volume ratio of reservoir and device volume.

In simple cases, a good approximation to the experimental curves can be achieved using analytical expressions. If the geometry is more complicated, appropriate numerical evaluations yield the necessary results. According to the laws of thermodynamics, a number of coupled processes modify the actual behavior, but as a rule in subordinate form. The influencing of the release kinetics by adsorption processes within the device and the films has died away after a short time and stationary kinetics determine the events.

For selection of the molecular species to be released, only the general conditions of stability over the desired release period apply. The active compounds coming into consideration are analogously restricted to those classes which are mentioned, for example, in DE 697 12 063, but are not restricted to these.

The segmented release device is used for the administration of molecules having a constant release rate to the human, animal or plant body. It can be used as a device in the particular bodies and, if necessary, taken to an accurately specifiable position within the body. It can be placed, for example, in the vicinity of the sites of action. After release of the entire molecular store or a proportion thereof fixed in another way, explanation can be carried out. Fresh filling in situ without explanation is likewise possible in a number of cases and can be achieved by means of the construction principles of the device.

The diffusion route between reservoir and surrounding medium defined by the expansion of the intersegment films determines the release rate of the molecules. The construction of the device allows a variety of geometrical embodiments which, by means of different local lengths of the intersegment films in one and the same device, leads to a specifiable direction-dependent diffusion rate. If the local distance of the reservoir via the intersegment films to the environment is smaller, the diffusion rate of the molecules into the outer medium is greater, and conversely.

### DESCRIPTION OF THE DRAWINGS

The drawings show only a few construction and functional principles and serve to illustrate the description better. Further embodiments can differ markedly therefrom in their geometry in adaptation to the particular requirements. The size ratios are shown differently from the real ratios in favor of the better presentation of the functional principle.

FIG. 1 shows an exploded representation of a segmented device.

FIG. 2 shows a sectional representation of a segmented device.

FIG. 3 shows an exploded representation of a further segmented device.

FIG. 4 shows a sectional view of a toroidal segmented device.

FIGS. 5A to 5D show sectional views of various embodiments of segmented devices.

FIG. 6 shows a sectional view of a segmented device with openings for filling thereof.

FIGS. 7A and 7B show a segmented device which was used for Experiments 1 and 2.

FIGS. 7C and 7D show release curves according to Example 1.

FIGS. 8A and 8B show release curves according to Example 2.

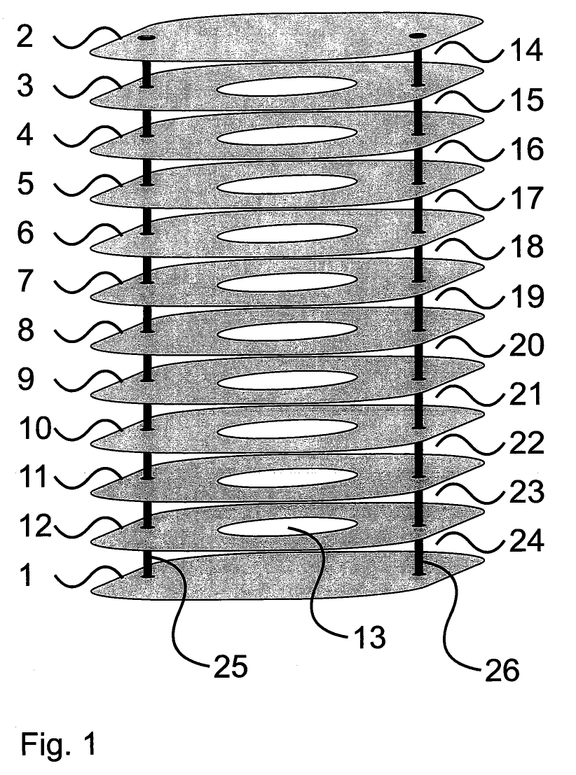

FIG. 1 in separated representation schematically shows a segmented, rectangular device having rounded comers and edges. Except for the base and cover segments which are closed with respect to the central areas, 1 and 2, all other segment discs 3 to 12 have a central opening or core drilling. The space 13, which is formed by the central openings and core drillings in the device, serves for the admittance of the molecules and thus forms a molecule reservoir. The intersegment films 14 to 24 between the discs serve for the diffusion of the active compound from the central reservoir into the surrounding medium. Connections 25 and 26 are part of the fixing of the device.

The filling of the device with the active substance can be carried out via reclosable openings. For example, this can take place via openings in cover and/or base discs with internal threads, into which a closure means, e.g. a fine threaded screw, is screwed. Cover and/or base discs can consist, for example, of titanium. The screwed-in closure means can additionally be sealed with wax or another sealing material.

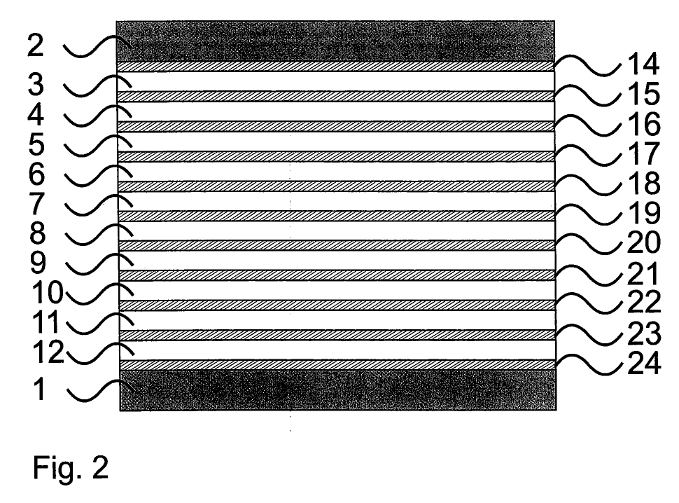

FIG. 2 shows the side view of a segmented release device. 1 and 2 represent the base and cover segments without an opening and core drilling. The disc segments are illustrated by 3 to 12 and 14 to 24 show the thin intersegment films, which serve for the exchange of the molecules with the environment. As a rule, the films are very much thinner than the segment discs.

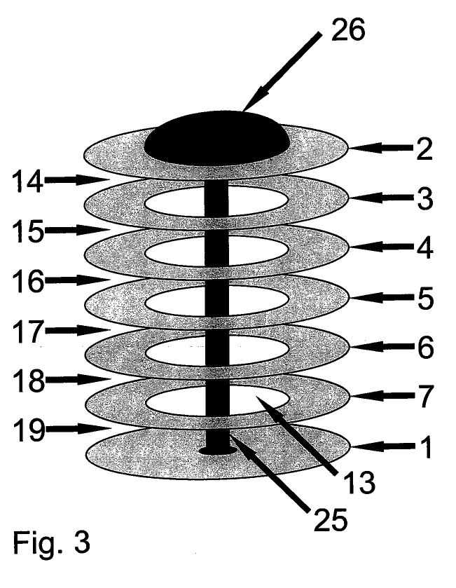

FIG. 3 shows a cylindrical segmented release device. 1 and 2 form the closing base and cover segments. 3 to 7 form the segment discs having a core drilling. 14 to 19 form the intersegment films between the segment discs. 25 and 26 are parts of the fixing and filling of the device and of the reservoir.

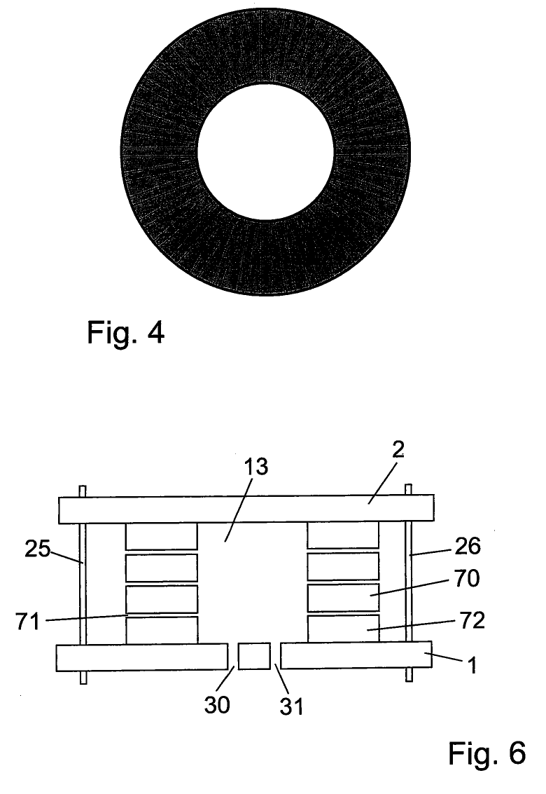

FIG. 4 shows a particular embodiment of the device without base and cover segments. The segment discs and intersegment films are closed to give a toroidal structure. In this case, the segment discs do not have a constant thickness.

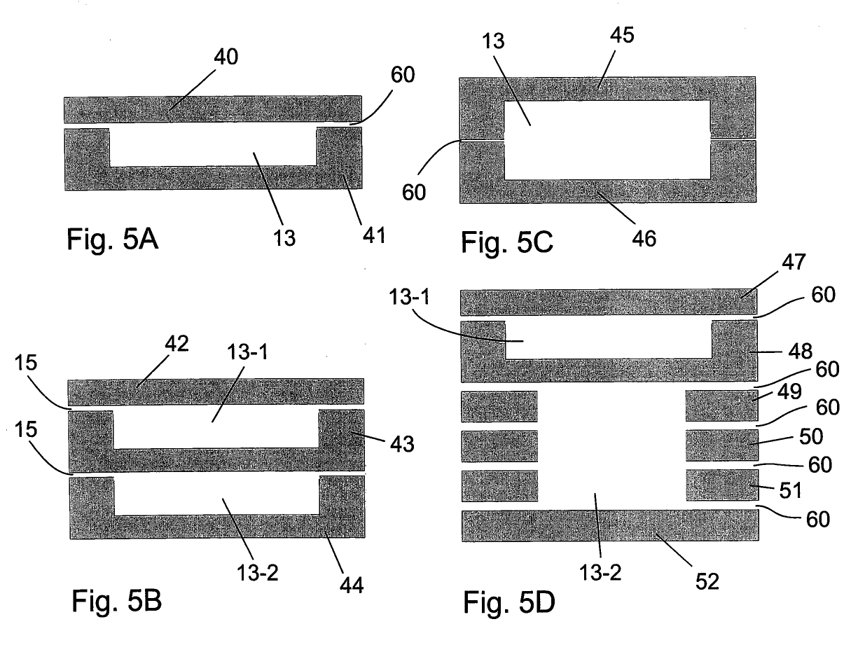

FIG. 5A shows a device consisting of a segment disc 41 and cover disc 40, segment disc 41 having a cavity 13 which forms the reservoir. An intersegment film 60 is situated between segment disc 41 and cover disc 41. FIG. 5B shows a structure of two segment discs 43, 44 and a cover disc 42, in each case an intersegment film 60 being arranged between all discs or being formed from the discs bordering on one another. The segment discs 43 and 44 in each case have a cavity 13-1 and 13-2, which form separate reservoirs. On the other hand, a segmented device of only two segment discs 45, 46 in each case having cavities facing one another for the formation of a reservoir 13 is shown in FIG. 5C. Finally, FIG. 5D shows a segmented device having a segment disc 48 with a cavity for the formation of a reservoir 13-1 and segment discs 49, 50 and 51 with a passage opening or core drilling for the formation of a second reservoir 13-2. Cover disc 47 and base disc 52 close the respective reservoirs.

FIG. 6 shows a release device having a first segment disc 72 having a passage opening, which is glued to a base disc 1. Further segment discs 70 are stacked on the first segment disc 72 with the interposition of intersegment films 71. Only three further segment discs were shown to be present, however considerably more segment discs, for example 15 to 20, can also be stacked one on the other. By means of a cover disc 2 and fixing means 25 and 26, the segment discs are pressed onto one another, such that a closed hollow space 13 results. This can subsequently be filled with the substance to be released via filling openings 30, 31. Finally, the filling openings are closed, for example, with fine-thread screws and sealed with wax.

Further advantageous embodiments are mentioned below, which can be realized individually or in any desired combination with one another:

Segmented device for the release of molecules, active compounds, medicaments, diagnostic, therapeutic and chemical reagents being understood thereunder, comprising:
stacked segment discs as segments with and without an internal opening or core drilling passing through or not passing through, which form connected or unconnected reservoirs for the molecules
permeable intersegment films between the segment discs through which the release of the molecules from the reservoir exclusively takes place
components for the holding and fixing of the device
   and which can be implanted in the human, animal or plant organism.
   The thickness and diameter of the segment discs and dimensions, shape and position of the internal opening or core drilling can vary.
   The segment discs can:
be circular discs or have the shape of conical sections and/or
have shapes which make the release of the molecules time- or direction-dependent according to specifiable requirements and/or
can have an outer diameter between 100 μm and 5 cm and/or
a thickness between 1 μm and 5 cm and/or
consist of permitted, biocompatible but not biodegradable materials or
consist of biocompatible and biodegradable materials.
   The device can contain at least two discs stacked one above the other and thus can contain at least one thin intersegment film communicating with the surroundings.
   Furthermore, the device can have cover and base discs and can have, arranged in between, any desired number of central discs which, with their stacking, form a connected or unconnected reservoir for molecule uptake.
   Preferred materials for the segment discs are—but not restricted thereto—ceramics, glasses, polymeric plastics, titanium, tantalum, steel, carbon modifications, silicon wafers, mica, inert inorganic solids, biomimetic hybrid materials.
   The segment discs can have unmodified surface roughnesses, which were produced by the preparation process.
   The segment discs can have modified surface roughnesses which are produced by polishing, grinding, cutting, fusing, coating or other surface treatment processes.
   The segment discs can have structured surface profiles which are produced by semiconductor technological or other interface chemical or physical processes.
   The segment discs can be structured by etching or lithographic processes.
   The segment discs can have roughnesses on the micrometre scale (1-50 μm) or nanometre scale (1-1000 nm). Roughnesses in the range less than 250 nm are preferred. For rough segment discs, an average segment disc interval of about 1 nm to 50 μm is advantageous. An interval of 2 nm to 20 μm and particularly preferably of 10 nm to 1 μm is preferred. The interval of the segment discs determines the thickness of the intersegment films (separate film or “hollow space”).
   The fixing components of the segmented device or means for the fixing and support of the segment discs can consist of permitted or biocompatible or inert materials.
   Between the segment discs, an intersegment film can be arranged which is formed either by surface roughnesses of the segment discs or from a separate film. This intersegment film forms a diffusion path between the reservoir and the surroundings of the device. The diffusion of the substances or molecules contained in the reservoir takes place exclusively through the intersegment film, such that the release is determined by the diffusion. Provided the segment discs are immediately one above the other, the intersegment film is formed by the surfaces of the segment discs having a certain roughness, which makes possible diffusion of the molecules contained in the reservoir between the segment discs. A roughness in the nanometre range is preferred in this case.
   The intersegment film between the segment discs can be filled, in particular if it is formed by rough segment disc surfaces, with physiological solution or the solution of the surrounding medium or a mixture of the reservoir medium and surrounding medium.
   The intersegment film can also be formed by a separate film, which is penetrable by the molecules or substances contained in the reservoir.
   In the case of microscale, preferably in the case of nanoscale, roughness, the intersegment film can be constructed and modified with multilayers of polymeric polyelectrolytes and/or nanoparticles prepared by sequential adsorption, according to the layer-by-layer process.
   In the case of nanoscale, preferably in the case of microscale, roughness, the intersegment film can be constructed and modified from polyelectrolyte complexes or from complexes of polyelectrolytes and nanoparticles, which are applied to the discs in any specifiable thickness in substance and are subsequently reduced to the desired intersegment film thickness on assembly and fixing of the segmented device by the mechanically specified pressure or by the structure of the device.
   The intersegment film can consist of a nanoscale and/or microscale porous and/or permeable intersegment film layer of a few nanometres up to some ten micrometres thickness (about 2 nm to about 20 μm, preferably 10 nm to 1 μm).
   The intersegment film can be constructed from organic, inorganic or hybrid materials which are not dissolvable or erodable or degradable in the biological medium, and permeable for the molecules from the inside outwards.
   The device can be adapted in size, shape, number of discs and intervals, intersegment film parameters and function to a required temporal and spatial release profile.
   The molecules contained in the reservoir are active compounds, medicaments, diagnostic, therapeutic and/or chemical reagents.
   The active compounds and medicaments can have a low, average or high molecular weight, and can be natural substances or synthetic substances.
   The molecules or substances in the reservoir can be diagnostic or chemical reagents, e.g. radioisotopes, radioactive compounds, fluorescent dyes and fluorescent dye-labelled chemical compounds and dissolved gaseous or readily volatile compounds.
   The molecules or substances contained in the reservoir can be filled with different formulations of the active compounds, medicaments, diagnostic, therapeutic and chemical reagents; these include solutions, saturated solutions in equilibrium with the solid substance, suspensions, emulsions, microemulsions, gels or solid matrices.
   The device can be filled with at least two substances in the reservoir.
   The device can consist of at least two reservoirs not connected to one another, which can be filled with substances of identical or different type.
   The device can be implanted in the human, animal or plant body and releases the substances or molecules with the temporal and spatial profile determined by the construction of the device.
   In the predominant part of the release period, the implanted device has a constant release rate.
   The device can be equilibrated with physiological solution or another medium before implantation.
   The device can be designed such that it can be refilled with substances in the human, animal or plant organism, i.e. that the device has means for refilling.
   All materials used for the devices can be produced, cleaned, sterilized, stored, treated, assembled, tested, adjusted and employed as an implant for the field of use implantation according to the prescribed pharmaceutical requirements, regulatory codes and guidelines.
   The devices can be used for
biotechnological purposes,
tissue engineering,
the release of substances in cell cultures,
bioreactors and ecosystems, and/or
biotechnological purposes
   with the release of biocides, with the release of substances which can induce, end or significantly modify reactions,
   with the release of labels, e.g. fluorescent dyes or radioactive substances, and
   with the release of gaseous or volatile substances, e.g. having hormonal character and/or
technical purposes
   with the release of biocides,
   with the release of substances which can induce, end or significantly modify reactions,
   with the release of labels, e.g. fluorescent dyes or radioactive substances which allow the monitoring of processes and
   with the release of gaseous or volatile substances.

### EXAMPLES

### Example 1

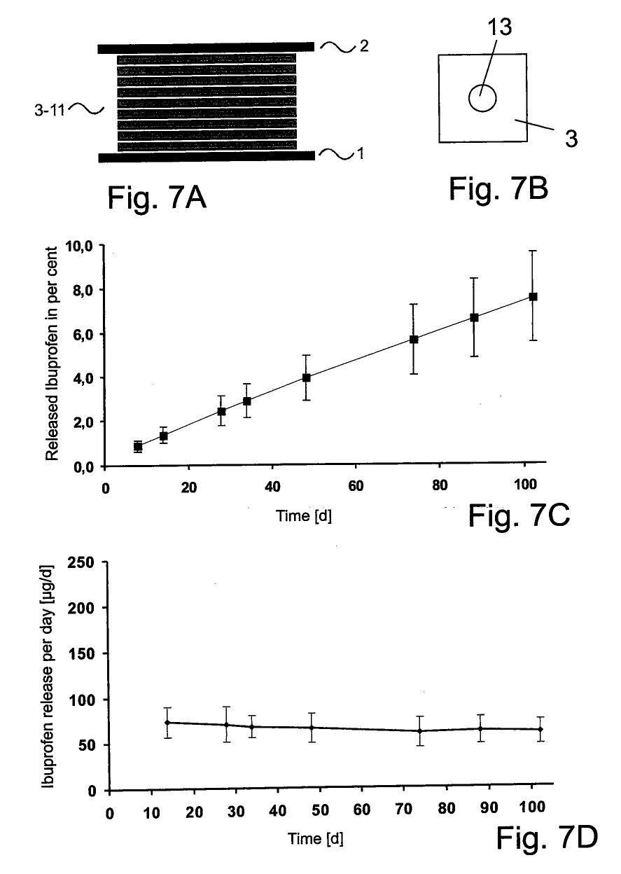

A release apparatus was produced from glass discs 3 to 11 (soda-lime glass, edge lengths 26.0×26.0 mm, disc thickness of 1.0 mm) having a surface roughness of 0.172-1.39 Ra (see FIGS. 7A and 7B). The release apparatus consisted of 9 discs (26.0×26.0 mm) having a core drilling 13 (diameter=8 mm) and was closed off on both sides by two discs without a core drilling. Separate intersegment films were not used. A reservoir volume of theoretically 452.4 μl results in the case of 9 discs having a core drilling and a disc thickness of 1.0 mm.

The release apparatus was filled with a model substance before closing (100 mg of crystalline ibuprofen) and the remaining reservoir volume was subsequently filled with release medium. Following this, the release behavior of the model substance was investigated at 37° C. in phosphate saline buffer pH 7.4 with addition of sodium azide.

FIGS. 7C and 7D show the ibuprofen release from the segmented glass apparatus having 10 intersegment films in phosphate saline buffer pH 7.4 with addition of sodium azide. As can be seen from FIG. 7D, the segmented release apparatus shows a constant release behavior of the model substance of about 65 μg/d for more than 100 days.

### Example 2

A release apparatus consisting of glass discs (soda-lime glass, edge lengths 26.0×26.0 mm, disc thickness of 1.0 mm) having a surface roughness of 0.172-1.39 Ra was produced (see FIGS. 7A and 7B), the discs being modified by a separating film covering only the outer edges of the discs such that an intersegment film thickness (hollow space) of about 50 μm was present. The release apparatus consisted of 9 discs 3 to 11 (26.0×26.0 mm) having a core drilling 13 (diameter=8 mm) and was closed off on both sides by two discs without a core drilling. Before closing, the release apparatus was filled with a model substance (100 mg of crystalline ibuprofen) and the remaining reservoir volume was subsequently filled with release medium. Following this, the release behavior of the model substance was investigated at 37° C. in phosphate saline buffer pH 7.4 with addition of sodium azide.

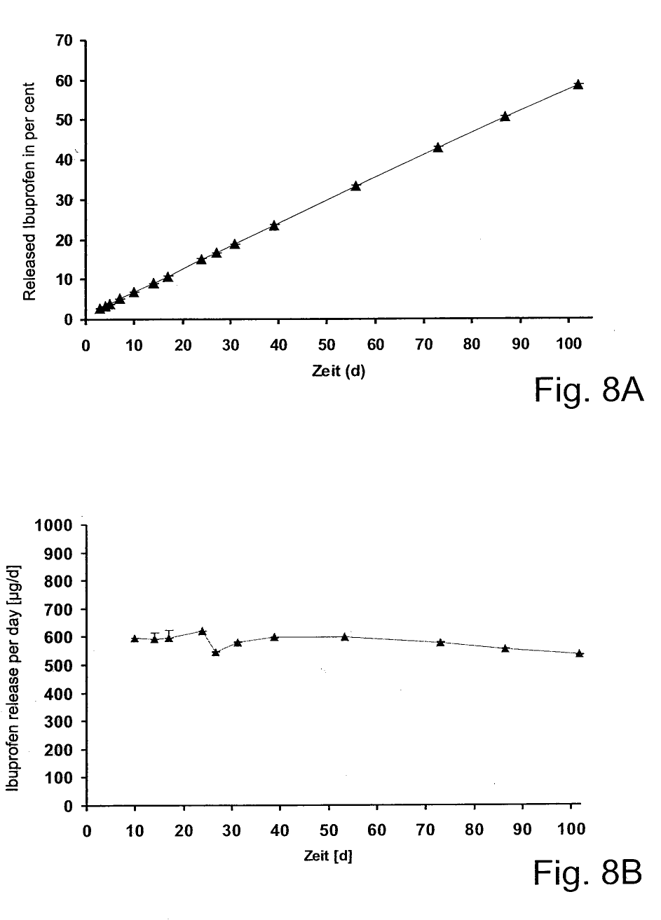

FIGS. 8A and 8B show the ibuprofen release from this segmented glass apparatus having 10 intersegment films and a modified intersegment film thickness in phosphate saline buffer pH 7.4 with addition of sodium azide. As can be seen from FIG. 8B, this segmented glass apparatus shows a release rate of the model substance of about 0.6 mg/d for 10 to 100 days.

### Example 3

A release apparatus was produced from round silicon discs (outer diameter 14.0 mm, thickness 0.525 mm) having a very low surface roughness. The release apparatus consisted of 15 discs having a core drilling (diameter=8 mm), a silicon disc without a core drilling as a cover plate and a base plate having closable openings for filling. A silicon disc having a core drilling was glued onto the base plate of titanium. The release apparatus was assembled underwater and fixed by means of a holder. Subsequently, the reservoir was emptied through the reclosable openings in the base plate before the release apparatus was filled with an enalaprilate suspension. The release apparatus thus had 15 intersegment films and showed a release rate of about 150 μg of enalaprilate per day.

### Example 4

As in Example 3, a release apparatus was produced from round silicon discs (outer diameter 14.0 mm, thickness 0.525 mm), the silicon discs being coated with polyelectrolyte multilayers. Before the assembly of the release apparatus, the silicon discs were coated with a 16 PAH/PSS layer. The release apparatus thus had 15 modified intersegment films and showed a release rate of about 75 μg of enalaprilate per day.

### Example 5

As in Example 4, a release apparatus was produced from round coated silicon discs (outer diameter 14.0 mm, thickness 0.525 mm), 25 discs having a core drilling being used. The release apparatus thus had 25 modified intersegment films and showed a release rate of about 140 μg of enalaprilate per day.

1. A segmented device for the release of molecules or substances, comprising:
at least two segment discs stacked one above the other having at least one cavity or passage opening, which forms at least one reservoir for the molecules or substances;
at least one permeable intersegment film between the segment discs, through which the release of the molecules or substances from the reservoir takes place exclusively; and
means for holding and fixing the segment discs.

2. The device according to claim 1, wherein the segment discs are circular discs.

3. The device according to claim 1, wherein the segment discs have the shape of conical sections.

4. The device according to claim 1, wherein the segment discs have outer diameters between 100 μm and 5 cm.

5. Device The device according to claim 1, wherein the segment discs have a thickness between 1 μm and 5 cm.

6. Device The device according to claim 1, further comprising a cover disc and a base disc, between which are arranged the segment discs, such that the stacked segment, cover, and base discs form the reservoir for the molecules or substances.

7. The device according to claim 1, wherein the segment discs being are stacked directly one on top of the other and their surfaces have a roughness such that the intersegment film is formed by means of the rough surfaces of the segment discs lying one above the other, which allows diffusion of the molecules from the reservoir outwards.

8. The device according to claim 7, wherein the roughness of the segment disc surfaces is on the micrometer scale.

9. The discs according to claim 7. wherein the roughness of the segment disc surfaces is on the nanometer scale.

10. The device according to claim 7, wherein the intersegment film is filled with a reservoir medium.

11. The device according to claim 1, wherein the permeable intersegment film between the segment discs comprises a different material than the segment discs.

12. The device according to claim 11, wherein the intersegment film having comprises layer-by-layer (LbL) multilayers of polymeric polyelectrolytes and/or nanoparticles.

13. The device according to claim 1, wherein the intersegment film has a thickness ranging from a few nanometers to tens of micrometers.

14. The device according to claim 1, wherein the molecules or substances are active compounds or pharmaceuticals.

15. The device according to claim 1, wherein the molecules or substances are diagnostic or chemical reagents.

16. The device according to claim 1, wherein the molecules or substances in the reservoir are present in a suspension, a saturated solution, a gel, an emulsion, a microemulsion, and/or solid matrices (tablets).

17. The device according to claim 1, wherein the reservoir is filled with at least two different substances.

18. The device according to claim 1, wherein the cavity or passage openings of the segment discs are shaped such that at least two reservoirs which are not connected to one another are formed.

19. The device according to claim 1, having means for refilling.

20. A method for the production of a segmented device for the release of molecules or substances, comprising:
producing at least two segment discs;
forming at least one cavity or passage opening in the segment discs;
stacking and fixing of the segment discs with an intersegment film between the segment discs, such that at least one inner reservoir is formed by the cavity or passage opening; and
filling the reservoir with the molecules or substances to be released.

21. The method according to claim 20, wherein the segment discs have a specified surface roughness.

22. The method according to claim 21, wherein the surface roughness of the segment discs is produced by polishing, grinding, cutting, fusing, coating, or other surface treatment processes.

23. The method according claim 20, wherein surface profiles are produced in surfaces of the segment discs.

24. The method according to claim 23, wherein the surface profiles are produced by etching or lithographic processes.

25. The method according to claim 20, wherein the intersegment film between the segment discs comprises a different material than the segment discs.

26. The method according to claim 25, further comprising bringing the material of the intersegment film to between the segment discs to the desired intersegment film thickness subsequently in the stacking and fixing of the segment discs by pressing of the segment discs.

27. The method according to claim 25, wherein the intersegment film is formed by application of alternately charged layers of molecules to the surfaces of the segment discs.

28. The method according to claim 25, wherein the intersegment film comprises polyelectrolyte complexes or complexes of polyelectrolytes and nanoparticles.

29. The method according to claim 20, wherein the device is implanted into a human, animal, or plant body, and the molecules or substances contained in the reservoir are released.

30. The method according to claim 20, wherein the device is equilibrated before implantation with a physiological solution or another medium.

31. (canceled)
